(19) United States
(12) Patent Application Publication
(10) Pub. No.: US 2025/0262240 A1
(43) Pub. Date: Aug. 21, 2025

(54) PHARMACEUTICAL COMPOSITIONS AND RELATED METHODS

(71) Applicant: Murata Manufacturing Co., Ltd., Nagaokakyo-shi (JP)

(72) Inventors: Yuki KIMURA, Nagaokakyo-shi (JP); Akimaro YANAGIMACHI, Nagaokakyo-shi (JP); Takayuki KONO, Nagaokakyo-shi (JP); Yusuke KOBAYASHI, Yokohama-shi (JP); Keita TSUBAKIMOTO, Nagaokakyo-shi (JP); Taiki ORITO, Nagaokakyo-shi (JP)

(21) Appl. No.: 19/198,642

(22) Filed: May 5, 2025

Related U.S. Application Data
(63) Continuation of application No. PCT/JP2023/038555, filed on Oct. 25, 2023.
(60) Provisional application No. 63/423,608, filed on Nov. 8, 2022.

(51) Int. Cl. A61K 33/24

(52) U.S. Cl. CPC A61K 33/24

(57) ABSTRACT

The disclosure provides pharmaceutical compositions comprising one or more nanofibers and/or nanoflakes of a material represented by the formula: MQₐO_{b}, wherein “M” is at least one element selected from the group consisting of Groups 3, 4, 5, 6, or 7; “Q” is at least one element selected from the group consisting of Groups 12, 13, 14, 15, or 16, provided that oxygen is excluded’; “a” is 0 or more and 2 or less; and “b” is greater than 0 and 2 or less. In some aspects, methods of treatment and prophylactic methods, based on the same, are also provided.

### CROSS-REFERENCE TO RELATED APPLICATION

The present application is a continuation of International Application No. PCT/JP2023/038555, filed on Oct. 25, 2023, which claims priority to U.S. Provisional Application No. 63/423,608, filed Nov. 8, 2022, the entire contents of each of which are incorporated herein by reference.

### TECHNICAL FIELD

The present disclosure relates to pharmaceutical compositions, adsorption methods, therapeutic methods, and prophylactic methods.

### BACKGROUND

An increasing number of patients are being diagnosed with renal disease each year, and according to the statistics of the Japanese Society for Dialysis Therapy, the number of chronic dialysis patients has increased by approximately 17% between 2010 and 2020. When renal functionality decreases due to a renal disease, the harmful (e.g., toxic) substances accumulate in the blood, and as a result, uremia, electrolyte metabolism abnormality, autoimmune disease, and other similar diseases and conditions may result. In order to treat the various symptoms associated with a renal disease, hemodialysis or another substitute for renal functionality is required to remove disease-causing substances; absent treatment, patients suffering from renal impairment will often develop life-threatening conditions (e.g., due to the build-up of toxic compounds in the subject's blood), eventually resulting in death. Hemodialysis, and other similar techniques, are often costly and detrimental to the quality of life of patients.

Hemoperfusion columns are commercially available and used as a filter for hemodialysis. One such column is marketed under the brand “Lixelle” by Kaneka Corporation, and comprises an adsorption-type blood purifier using cellulose beads on which cetylamine is immobilized as an adsorption body.

As an alternative for hemodialysis, orally ingested absorbents may be used. Such compounds are designed to adsorb one or more toxic substances in the body, so that they may be discharged along with the adsorbent after it passes through the subject's digestive tract. For example, medicinal charcoal is a well-known adsorbent of toxic compounds (e.g., poison) and gas in the digestive tract.

Pharmaceuticals based on carbon-based adsorbents have been developed, such as Kremezin (marketed by Mitsubishi Tanabe Pharma Corporation), which comprises spherical particles of coal. Kremezin is known to be capable of adsorbing uremic toxins present in the gastrointestinal tract without being absorbed in the body and being excreted with feces. See, e.g., Yoshiteru Honda et al., “Study on Adsorption Property of Spherical Adsorption Coal (Kremezin Active Material)”, Journal of the Nippon Hospital Pharmacists Association, 1997, Vol. 23, No. 3, 219-224.

Additional orally administered adsorbents include formulations of Kalimate (e.g., “Kalimate Powder,” “Kalimate Dry Syrup 92.59%,” and “Kalimate Oral Solution 20%,” marketed by Kowa Company Ltd.). Kalimate is a calcium-type cation exchange resin capable of exchanging potassium ions in the intestinal tract with calcium ions in the structure to reduce the blood potassium value. Adsorbents have been developed to target other compounds, such as Phosblock (marketed by Kyowa Kirin Co., Ltd.) which is a polycationic polymer that can be directly excreted in feces without being absorbed by binding to phosphate ions released from food in the gastrointestinal tract.

### BRIEF SUMMARY

While several options are available to treat renal insufficiency or failure (e.g., hemodialysis, and the use of adsorbent agents), known techniques suffer from a variety of shortcomings that limit their effectiveness. For example, hemoperfusion columns require complex and expensive equipment. Known adsorbents compounds, e.g., medicinal coal (and pharmaceuticals based on the same) are limited to capturing adsorbates present in the gastrointestinal tract by ingestion, enterohepatic circulation, production via enterobacteria, or the like. Moreover, known adsorbents are limited to targeting specific types of adsorbates.

The present disclosure provides novel pharmaceutical compositions capable of adsorbing various disease-causing substances in vivo, as well as methods of using the same to adsorb disease-causing substances. Such methods may be used, e.g., as a therapeutic method, or as prophylactic method, in order to treat (or prevent) renal insufficiency or failure, among other conditions.

In a first general aspect, the disclosure provides a pharmaceutical composition comprising one or more nanofibers and/or nanoflakes of a material represented by the following formula:

MQₐO_{b}
wherein “M” is at least one element selected from the group consisting of Groups 3, 4, 5, 6, or 7,
wherein “Q” is at least one element selected from the group consisting of Groups 12, 13, 14, 15, or 16, provided that oxygen is excluded,
wherein “a” is 0 or more and 2 or less,
wherein “b” is greater than 0 and 2 or less, and
wherein a crystal structure of the material is optionally a lepidocrocite type.

In some aspects, the material has a peak at a diffraction angle 20 in a range of 2° or more and 12° or less in an X-ray diffraction pattern.

In some aspects, the “Q” is C, and the “a” is not 0.

In some aspects, the “M” is Ti, the “Q” is C, and the “a” is not 0.

In a second general aspect, the disclosure provides a method for adsorbing a disease-causing substance in vivo, in a subject in need thereof, comprising: administering the pharmaceutical composition according to claim 1 to the subject, thereby reducing the amount or concentration of the disease-causing substance within the subject, upon excretion of the composition according to claim 1 from the subject.

In some aspects, the disease-causing substance comprises a uremic substance having a molecular weight of 100 or more.

In some aspects, the uremic substance having a molecular weight of 100 or more comprises β2-microglobulin.

In some aspects, the disease-causing substance comprises an electrolyte.

In some aspects, the electrolyte comprises Na and/or K.

In some aspects, the disease-causing substance comprises a cytokine, and may optionally comprise an interleukin, an interferon, a chemokine, a hematopoietic factor, a cell growth factor, and/or a tumor necrosis factor.

In some aspects, the composition is formulated as an oral dosage form.

In a third general aspect, the disclosure provides a method of treatment, comprising: administering, to a subject in need thereof, an effective amount of nanofibers and/or nanoflakes of a material represented by the following formula:

MQₐO_{b}
wherein “M” is at least one element selected from the group consisting of Groups 3, 4, 5, 6, or 7,
wherein “Q” is at least one element selected from the group consisting of Groups 12, 13, 14, 15, or 16, provided that oxygen is excluded,
wherein “a” is 0 or more and 2 or less,
wherein “b” is greater than 0 and 2 or less, and
wherein a crystal structure of the material is optionally a lepidocrocite type;
thereby reducing a concentration or amount of one or more disease-causing substances in the subject upon excretion of the nanofibers and/or nanoflakes from the subject.

In some aspects, the material has a peak at a diffraction angle 20 in a range of 2° or more and 12° or less in an X-ray diffraction pattern.

In some aspects, the one or more symptoms associated with the renal disease comprise hypernatremia and/or hyperkalemia.

### BRIEF DESCRIPTION OF THE DRAWINGS

FIG. 1 shows an XRD pattern of a material (TiCO) produced in Example 1.

### DETAILED DESCRIPTION

The present disclosure provides novel pharmaceutical compositions, e.g., pharmaceutical compositions capable of adsorbing a disease-causing substance in vivo, as well as methods of using the same to adsorb toxic or otherwise undesirable compounds, e.g., as a therapeutic treatment to reduce one or more symptoms caused by renal insufficiency or failure, and other medical conditions. In some aspects, the pharmaceutical compositions described herein may be administered as a prophylactic to prevent or delay the onset of renal failure or dysnfunction, or the development of symptoms related to the same.

Without being bound to a theory, the pharmaceutical compositions of the present disclosure are believed to be useful for the treatment and/or prevention of various diseases because they contain nanofibers and/or nanoflakes of a material represented by MQₐO_{b} (which in the present disclosure is also simply referred to as “MQO”) and can adsorb one or more substances (e.g., disease-causing substances).

In vivo, there are main two pathways in place to remove disease-causing substances such as a metabolite, a waste product, a harmful substance, and an excessive electrolyte. Such compounds may be excreted in the gastrointestinal tract from the bile duct via the duodenum (being contained in bile in the liver as bile excretion), or excreted in urine via glomerular filtration in the kidney. However, in a patient suffering from renal failure, the excretion function of metabolites and the like by the kidneys is not sufficient, and periodic treatment by dialysis therapy (hemodialysis, peritoneal dialysis, etc.) is required, resulting in significant deterioration of quality of life.

Without being bound to a specific theory, MQO contained in the pharmaceutical compositions of the present disclosure has the ability to effectively adsorb disease-causing substances and the like, and thus it is expected that when it is orally administered, the disease-causing substances in the blood can be adsorbed and eliminated from the blood through mutual access between the intestinal tract and the blood vessels while staying in the intestinal tract. In addition, by adsorbing a substance that can be converted into a harmful substance after being absorbed into blood from the intestinal tract, it is expected to adsorb and eliminate a harmful influence on the body in advance. Since it is considered that MQO adsorbed with the disease-causing substance is not absorbed from the intestinal tract, it is expected that MQO passes through the gastrointestinal tract and is excreted as it is together with feces. As described above, while two endogenous excretion pathways are available to remove toxic compounds (e.g., bile excretion and urine excretion), pharmaceutical compositions of the present disclosure can be said to provide a third metabolic pathway, and the use of such compositions is expected to lead to reduction of treatment related to dialysis therapy in patients with renal failure, and other conditions related to or caused by renal failure or insufficiency. In addition, it is expected that MQO-based compositions also adsorb disease-causing substances and the like contained in the contents of the diet in the gastrointestinal tract and do not cause intestinal tract absorption.

In some aspects, the pharmaceutical compositions of the present disclosure contain a material represented by the following Formula (1):

MQₐO_{b} (1)
wherein “M” is at least one element selected from the group consisting of Groups 3, 4, 5, 6 or 7, and may contain a so-called early transition metal, for example, at least one element selected from the group consisting of Sc, Ti, Zr, Hf, V, Nb, Ta, Cr, Mo and Mn, and preferably at least one element selected from the group consisting of Ti, V, Cr, Mo and Mn,
“Q” is at least one element (excluding oxygen) selected from the group consisting of Groups 12, 13, 14, 15, or 16, and may contain, for example, at least one element selected from the group consisting of B, C, N, Si, P, and S,
“a” is not less than 0 and not more than 2, and
“b” is more than 0 but not more than 2.

The pharmaceutical composition of the present disclosure is useful for the treatment and/or prevention of various diseases because it contains nanofibers and/or nanoflakes of a material represented by MQₐO_{b} and can adsorb a substance that can cause a disease (disease-causing substance).

Hereinafter, the predetermined material is also simply referred to as “MQO”. Examples of MQO include materials represented by formulas such as TiO₂, TICO, TiCON, VO₂, VCO, VCON, CrO₂, CrCO, CrCON, MoO₂, MoCO, MoCON, MnO₂, MnCO, and MnCON. For example, in Formula (1), “M” may be Ti, and the “Q” may be C. In some aspects, in Formula (1), “a” may not be 0.

MQO may typically have a peak in a range in which a diffraction angle 20 is 2° or more and 12° or less in an X-ray diffraction (XRD) pattern. Although the present disclosure is not bound by any theory, the fact that MQO has a peak in a range of 20=not less than 2° and not more than 12° in the XRD pattern is considered to mean that MQO has a crystal structure different from that of a well-known metal oxide.

In the present disclosure, an XRD pattern is a pattern (the vertical axis represents intensity, and the horizontal axis represents 2θ) obtained by θ-axis direction scanning with an XRD analyzer using CuKα rays (=about 1.54 Å) as characteristic X-rays, and may also be referred to as an “XRD profile”. The peaks in the XRD pattern can be identified visually or using a software used with the XRD analyzer. In order to measure an XRD pattern in a low angle range of 2θ as accurately as possible, it is preferable to install a c-axis oriented MQO membrane in an XRD analyzer (for example, as in the Examples described herein, a self-standing membrane obtained by removing a filter after suction filtration is disposed with the surface that had been in contact with the filter facing downward) to perform the measurement.

Without being bound to a theory, it can be considered that the crystal structure of MQO is an anatase type, a lepidocrocite type, or a mixture thereof. In some aspects, the crystal structure of MQO may be a lepidocrocite type.

MQO may be produced using a first raw material and a second raw material, for example, as follows. The first raw material contains at least “M,” the second raw material contains at least “Q,” and the first raw material and the second raw material can react in a protic solvent to generate MQO.

As the first raw material, a material represented by the following Formula (2) can be used:

M_{c}A¹_{d} (2)
wherein “M” is as described above,
where “A¹” is at least one element selected from the group consisting of Groups 12, 13, 14, 15, or 16, and may contain, for example, at least one element selected from the group consisting of B, C, N, O, Si, P, and S, and
where “c” and “d” are each independently not less than 1 and not more than 5.

In some aspects, the material represented by Formula (2) is different from the MQO in a given composition. Typically, the material represented by Formula (2) may not have a peak in a range in which a diffraction angle 20 is not less than 2° and not more than 12° in an X-ray diffraction (XRD) pattern.

Examples of the first raw material represented by Formula (2) include TiB₂, TiB, TIC, TiN, TiO₂, Ti₅Si₃, Ti₂SbP, VO₂, V₂O₄, NbC, Nb₂O₅, MoO₂, MoO₃, MoS₂, MnO₂, Mn₃O₄, and MnCO₃. In some aspects, MnO₂ that may be used as the first raw material has a peak in the vicinity of 2θ=13° and does not have a peak in the range where 2θ is not less than 2° and not more than 12° in the XRD pattern.

Alternatively, or in addition to the above, a material represented by the following Formula (3) (hereinafter, also simply referred to as “MAX phase” or “MAX raw material”) may be used as the first raw material:

MₘA²Xₙ (3)
wherein “M” is as described above,
“X” is at least one element selected from the group consisting of C and N,
“n” is not less than 1 and not more than 4,
“m” is more than n but not more than 5,
“A²” is at least one element selected from the group consisting of Groups 12, 13, 14, 15, or 16, and may be a Group A element, typically Group IIIA and Group IVA, and more particularly may contain at least one selected from the group consisting of Al, Ga, In, TI, Si, Ge, Sn, Pb, P, As, S, and Cd, and is preferably Al.

The MAX phase has a crystal structure in which a layer constituted by A² atoms is located between two layers represented by MₘXₙ (each X may have a crystal lattice located in an octahedral array of M). In the case of m=n+1, the MAX phase typically includes repeating units in which each one layer of X atoms is disposed in between adjacent layers of n+1 layers of M atoms (these are also collectively referred to as an “MₘXₙ layer”), and a layer of A² atoms (“A² atom layer”) is disposed as a layer next to the (n+1)th layer of M atoms. However, the MAX phase is not limited thereto.

Examples of the first raw material represented by Formula (3) include Ti₃AlC₂, Ti₃GaC₂, and Ti₃SiC₂.

As the first raw material, the material represented by Formula (2) and the material represented by Formula (3) may be used together (for example, as a mixture).

As the second raw material, an ion-binding substance having a carbon-containing group can be used. The ion-binding substance having a carbon-containing group contains C. Examples of the ion-binding substance include an ammonium salt, a phosphate salt, and a sulfate salt.

More specifically, a quaternary ammonium salt can be used as the second raw material. Examples of the quaternary ammonium salt include tetramethylammonium hydroxide (TMAH), tetraethylammonium hydroxide (TEAH), tetrapropylammonium hydroxide (TPAH), tetrabutylammonium hydroxide (TBAH or TBAOH), benzyltrimethylammonium hydroxide, tetrabutylammonium fluoride (TBAF), tetrabutylammonium chloride (TBACl), tetrabutylammonium bromide (TBAB), tetrabutylammonium iodide (TBAI), benzyltriethylammonium chloride (BTEAC), hexadecyltrimethylammonium bromide, cetyltrimethylammonium bromide (CTAB), benzetonium chloride, benzalkonium chloride, and cetylpyridinium chloride (CPC). Among them, TMAH and TBAOH are preferable.

Alternatively, or in addition to the above, other ion-binding substances containing P and/or S etc. may be used as the second raw material.

The protic solvent may be any solvent that can at least partially dissolve the first raw material and the second raw material, and may be particularly an aqueous solvent. As the protic solvent, water, an alcohol (For example, ethanol, 2-propanol, 2-propanol, isopropanol), a carboxylic acid (for example, acetic acid, formic acid), or the like is used. The aqueous solvent may be composed of water and optionally a liquid substance compatible with water (for example, a protic solvent other than water), and is preferably water.

The first raw material and the second raw material are reacted in the protic solvent. The second raw material can be added to the protic solvent in advance. The ratio of the second raw material to the total of the protic solvent and the second raw material may be, for example, 5% by mass or more, particularly 20% by mass or more, and/or may be, for example, 80% by mass or less, particularly 50% by mass or less. The first raw material can be further added to and mixed with the protic solvent to which the second raw material has been added. In such a mixture, a reaction for producing MQO proceeds. The temperature (reaction temperature) of the mixture (which may contain the reaction product) may be, for example, 15° C. or higher, particularly 40° C. or higher, and/or, for example, 100° C. or lower, particularly 80° C. or lower. A mixing time (reaction time) may, for example, be 1 day or more, particularly 2 days or more, and/or may, for example, be 10 days or less, particularly 7 days or less. The mixing may be carried out, for example, using a hot plate stirrer. However, the treatment operation and conditions (temperature and time and the like) under which the reaction can proceed are not limited to the above, and may be appropriately selected according to the first raw material, the second raw material, and the protic solvent and the like to be used.

By the above reaction, MQO is generated, and can eventually grow into nanofibers of MQO, and further into nanoflakes of MQO. Without limiting the present disclosure, the resulting nanofibers of MQO may be in the form of two-dimensionally extending nanoribbons. A plurality of nanofibers (for example, nanoribbons) of MQO may be bonded and/or integrated with each other to grow into nanoflakes two-dimensionally extending. A plurality of MQO nanoflakes may overlap each other (for example, by van der Waals force) to form a laminate. Although the present disclosure is not bound by any theory, such generation and growth of MQO can be considered to be due to a bottom-up type synthesis reaction (see, for example, Hussein O. Badr, et al., “Bottom-up, scalable synthesis of anatase nanofilament-based two-dimensional titanium carbo-oxide flakes,” Materials Today (2021)).

In the present disclosure, MQO is a solid. MQO may be formulated as a particle or powder.

The mixture after the reaction (also referred to as a reaction mixture) may be subjected to post-treatment. Examples of the post-treatment include washing, impact application (including shear force application), drying (for example, freeze dry, heat dry), and pulverization.

The washing may be performed using a protic solvent. The same description as above may apply to the protic solvent, and the protic solvent may be washed with, for example, water or an alcohol. After washing, a separation operation (centrifugation and/or decantation) may be performed. The washing and separation operations may be repeated until the pH of a supernatant liquid after centrifugation is, for example, 8 or less.

Optionally, washing may be performed using an aqueous solution of a metal salt instead of or in addition to the above washing. The metal salt may be, for example, a halide (fluoride, chloride, bromide, or iodide) of an alkali metal (Li, Na, or K or the like), typically LiCl, NaCl, or KCl or the like. Specifically, for example, washing may be performed using an aqueous solution of a metal salt having a molar concentration of 1 to 10. After washing, a separation operation (centrifugation and/or decantation) may be performed. Also in this case, the washing and separation operations may be repeated as necessary until the pH of the supernatant liquid after centrifugation is, for example, 8 or less.

During and/or after washing, an impact such as vibration and/or ultrasound may be applied. This makes it possible to promote the dispersion or the like of MQO particles (for example, nanofibers/nanoflakes, and so on). When the MQO particles are aggregated, they can be crushed. Such an effect is remarkably obtained when an impact is applied during washing using an aqueous solution of a metal salt (it is considered that metal cations derived from the metal salt can enter gaps of the aggregates and the aggregates can be crushed). The impact can be imparted using, for example, any one or more of a handshake, an automatic shaker, a mechanical shaker, a vortex mixer, a homogenizer, and an ultrasonic bath and the like.

Since the MQO particles are solid, a separation operation may be performed at any suitable timing to remove unwanted liquid components if present. As a final separation operation, for example, a drying operation, typically freeze drying or heat drying, may be performed. The freeze-drying may be performed, for example, by freezing a mixture containing MQO particles and a liquid component at any suitable temperature (for example, −40° C.), followed by drying under a reduced pressure atmosphere. The heat drying can be performed, for example, by drying a mixture containing MQO particles and a liquid component at a temperature of 25° C. or higher (for example, 200° C. or lower) under a normal pressure or a reduced pressure atmosphere. The pulverization is not particularly limited, but can be performed using, for example, a combination of a mortar and a pestle, or an IKA mill or the like. The pulverization may be performed after drying.

As described above, it is possible to obtain the pharmaceutical composition of the present disclosure containing MQO particles (nanofibers and/or nanoflakes). Although MQO particles are represented by Formula (1), the particle of MQO does not need to include only the constituent element of Formula (1). Although the present disclosure is not limited, MQO particles may optionally have at least one selected from the group consisting of a hydroxyl group, a chlorine atom, an oxygen atom, a hydrogen atom, and a nitrogen atom as the modification or termination T present on the surface. In addition, MQO particles may have two or more layers, and at least one selected from the group consisting of ammonium ions (for example, quaternary ammonium cations) and metal cations (for example, alkali metal ions and alkaline earth metal ions) may be present between these layers.

The BET specific surface area of MQO particles is not particularly limited, but may be, for example, 10 m²/g or more and 500 m²/g or less. The BET specific surface area is calculated using a BET equation from the isothermal adsorption curve of nitrogen gas or other gases at a liquid nitrogen temperature (77 K) by an adsorption method with the nitrogen gas or the other suitable gases (such as krypton (Kr) gas).

The particle diameter of the MQO particles may be, for example, 0.01 nm or more, particularly 0.1 nm or more, and 1 nm or more, and/or may be, for example, less than 1000 nm, particularly 100 nm or less, and 50 nm or less. Such particles may also be referred to as nanoparticles.

The morphology of MQO particles may be at least one selected from the group consisting of nanofibers, nanoflakes or a laminate of nanoflakes. The nanoflakes and the laminate of nanoflakes comprise two-dimensional materials.

The nanofibers of the present disclosure may also be referred to as nanowires. The term “nanofiber,” as used herein, means a solid material extending in the longitudinal direction, and an outer dimension in a cross section perpendicular to the longitudinal direction is on the nano order (that is, 1 nm or more and less than 1000 nm) or on the sub-nano order smaller than the nano order (less than 1 nm, for example, 0.1 nm or more and less than 1 nm). The cross-sectional outer dimension of the nanofiber may be, for example, 0.1 nm or more, and particularly 1 nm or more, and may be, for example, 100 nm or less, particularly 50 nm or less, and preferably 15 nm or less.

The nanoflakes of the present disclosure may also be referred to as a “nanosheet” or as a two-dimensional (nano) sheet. The term “nanoflake” means a solid having a two-dimensionally extended surface and having a relatively small thickness with respect to the largest dimension of the surface, wherein the thickness is on the nano order or on the sub-nano order smaller than the nano order. The thickness of one layer of the nanoflake may be, for example, 0.01 nm or more, particularly 0.8 nm or more and, for example, 20 nm or less, particularly 3 nm or less. The largest dimension (which may correspond to the “in-plane dimension” of the particle) in a plane (two-dimensional sheet plane) parallel to the layer of nanoflakes may be, for example, 0.1 μm or more, in particular 1 μm or more and, for example, 200 μm or less, in particular 40 μm or less. The nanoflake can be constituted by the aggregation of nanofibers.

A stack of nanoflakes according to the present disclosure may also be referred to as a “multilayer” MQO. A distance (interlayer distance or void dimension) between two adjacent nanoflakes (or MQO of two adjacent layers) is not particularly limited.

Each dimension described above can be obtained as a number average dimension (number average of at least 40) based on a photograph observed with a scanning electron microscope (SEM), a transmission electron microscope (TEM), or an atomic force microscope (AFM) (if necessary, processing is performed by a method such as a focused ion beam (FIB)), or a distance in a real space calculated from a position on a reciprocal lattice space of a (002) plane measured by an X-ray diffraction (XRD) method.

However, it should be noted that in the present disclosure, MQO is not limited to the above-described form, and may have any suitable form.

Pharmaceutical compositions of the present disclosure (containing MQO particles) may contain unreacted first raw material and/or second raw material as impurities, and may contain a substance derived from the first raw material, the second raw material and/or the protic solvent. For example, when a quaternary ammonium salt is used as the second raw material, N may present (remain) in arbitrary form in MQO particles. For example, when the MAX raw material is used as the first raw material, in the present disclosure, MQO particles may contain a relatively small amount of remaining A atoms, for example, 10% by mass or less with respect to the original A atoms. The remaining amount of A atoms can be preferably 8% by mass or less, and more preferably 6% by mass or less. However, even if the remaining amount of A atoms exceeds 10% by mass, there may be no problem depending on the use conditions or the like.

In order to obtain MQO particles with higher purity, it is preferable to repeat washing and centrifugation multiple times and to recover the supernatant liquid after final centrifugation. Such a supernatant liquid can be formed into a slurry containing MQO particles as it is, appropriately diluted with a liquid medium, or mixed with a liquid medium after drying.

The intermediate and the target product in the production method described above may be isolated by a commonly used purification method. Examples the purification method includes suction filtration; and drying such as heat drying, freeze drying, and vacuum drying.

MQO (for example, MQO particles) exhibits an action of adsorbing a disease-causing substance such as an electrolyte, a protein having a moderate molecular weight, particularly a uremic substance in an adsorption test using human plasma. In other words, the pharmaceutical composition containing MQO can be used for adsorbing a disease-causing substance in vivo. The disease-causing substance may contain an electrolyte, and more specifically, may contain at least one selected from the group consisting of Na and K. Alternatively or in addition, the disease-causing substance may contain a uremic substance having a molecular weight of 100 or more Daltons, and more specifically, may contain β2-microglobulin or the like. Examples of the disease-causing substance include parathyroid hormone and uremic proteins such as B2-microglobulin described above (specifically, a medium-molecular-type uremic substance); other proteins such as albumin and M protein. Alternatively, or in addition, the disease-causing substance may contain cytokines (In one embodiment, inflammatory cytokines such as interleukin 18, interleukin-6 (IL-6), interferon-γ (INF-γ), tumor necrosis factor (TNF-α)) such as interleukins, interferons, a chemokine, a hematopoietic factor, a cell growth factor, and a tumor necrosis factor. The inflammatory cytokine can cause various inflammatory symptoms in vivo, and an effect of preventing inflammation is expected by adsorbing them. The disease-causing substance may contain, for example, an enzyme such as α-amylase as a substance whose blood concentration increases due to a decrease in renal function.

Thus, pharmaceutical compositions containing MQO are useful for the treatment or prevention of a disease. Examples of the disease include various symptoms associated with a renal disease (may also be referred to as kidney disease, renal dysfunction) such as acute kidney disease (including acute renal failure) and chronic kidney disease (including chronic renal failure and end-stage renal failure), particularly various symptoms associated with renal failure. In other words, methods for treating or preventing various symptoms associated with a renal disease, including administering an effective amount of MQO to a subject, can be performed. Specific examples of the various symptoms associated with a renal disease include electrolyte metabolic disorders such as hypernatremia and/or hyperkalemia.

Furthermore, since MQO can predominantly adsorb the disease-causing substance, it is expected that the disease-causing substance is adsorbed in vivo and the disease-causing substance in the blood is reduced by administering MQO to the subject. In other words, an adsorption method including administering an effective amount of MQO to a subject to adsorb a disease-causing substance in vivo can be performed. In particular, it is expected that the disease-causing substances in the blood can be adsorbed and eliminated from the blood through mutual access between the intestinal tract and the blood vessel while staying in the intestinal tract by oral administration. In addition, it is also expected that reduction of the disease-causing substances in the blood leads to reduction of treatment related to dialysis therapy and reduction of the burden on organs such as the kidney.

The pharmaceutical compositions according to the present disclosure may be formulated in various dosage forms depending on usage. Examples of such a dosage form include powder, a granule, a fine granule, a dry syrup, a tablet, a capsule, liquid, and a sublingual agent, and also include an injection, an ointment, a suppository, and a patch.

The pharmaceutical compositions according to the present disclosure may be formulated as a pharmaceutical composition containing MQO as an active ingredient and one or more pharmacologically acceptable additives, using known methods compatible with the preparation of a desired dosage form. Examples of such additives include an excipient, a disintegrant, a binder, a lubricant, a diluent, a buffering agent, an isotonizing agent, a preservative, a wetting agent, an emulsifying agent, a dispersing agent, a stabilizing agent, and a solubilizing agent. The pharmaceutical compositions of the present disclosure may be prepared by appropriately mixing MQO and the additive or diluting and dissolving MQO with an additive.

The pharmaceutical compositions according to the present disclosure may be administered systemically or locally, orally or parenterally (nasal, pulmonary, intravenous, intrarectal, subcutaneous, muscle, transdermal). In one aspect, the pharmaceutical composition according to the present disclosure may be administered orally.

When the pharmaceutical composition of the present disclosure is used for treatment, the dose of MQO as an active ingredient thereof is appropriately determined depending on the age, sex, weight, disease, degree of treatment, and the like of the patient. For example, in the case of oral administration, the dose may be appropriately administered once or in several divided doses in the range of about 100 mg to 10 g/body per day of an adult (body weight of 60 kg) as an effective amount.

In addition, a pharmaceutical composition containing MQO may be used for producing a medicine for treating or preventing a disease.

### EXAMPLES

The present disclosure will be described more specifically with reference to the following examples, but the present disclosure is not limited thereto.

### Example 1

### Preparation of TiCO

First, a vessel (100 mL I Boy) was charged with 10 g of titanium carbide (TiC, manufactured by Kojundo Chemical Laboratory Co., Ltd.) and 30 mL of a 25 mass % aqueous tetramethylammonium hydroxide (TMAH) solution (manufactured by Tokyo Chemical Industry Co., Ltd.). Thereto was placed a stirrer chip having a length substantially equal to the inner diameter of the circular bottom surface of the container (35 mm). While the container was kept at 50° C. in a water bath, the mixture in the container was stirred with the stirrer chip and maintained for 120 hours, thereby allowing the reaction to proceed. Next, the reaction mixture in the container was transferred to a 50 mL centrifuge tube with a stainless steel spatula (without the addition of a liquid medium such as ethanol or water). Centrifugation was performed using a centrifuge under conditions of 3500 G and 5 minutes to precipitate the solid content. (i) After centrifugation, the supernatant liquid was discarded, (ii) 40 mL of ethanol (manufactured by FUJIFILM Wako Pure Chemical Corporation) was added to the remaining precipitate in the centrifuge tube, and the mixture was subjected to handshake for 5 minutes (reslurry), and (iii) centrifugation was performed under the same conditions as described above. The operations (i) to (iii) were repeated until the pH of the supernatant liquid was 8 or less. When the operations were repeated three times, the pH of the supernatant liquid became 8 or less. Therefore, this supernatant liquid was discarded, and the repeated operations were terminated. 40 mL of pure water was added to the remaining precipitate in the centrifuge tube, and the mixture was shaken and stirred for 15 minutes using an automatic shaker. Thereafter, centrifugation was performed using a centrifuge under the conditions of 3500 G and 30 minutes, and the supernatant liquid was recovered as a sample slurry. The resulting sample slurry corresponds to a slurry including TICO (which may have the form of nanofibers and/or nanoflakes) (see analytical results below).

### Analysis

The sample slurry prepared above was filtered with suction overnight using a nutsche. As a filter for suction filtration, a membrane filter (Durapore, pore diameter 0.45 μm, manufactured by Merck Corporation) was used. After suction filtration, a precursor membrane on the filter was dried overnight at 80° C. in a vacuum oven, and the filter was removed to obtain a free-standing membrane.

The free-standing membrane thus obtained in the same manner as described above was analyzed by X-ray photoelectron spectroscopy (XPS). Peaks corresponding to Ti 2p, C 1s, O 1s, and N 1s were observed in the obtained XPS spectrum, and thus Ti, C, O, and N were detected. Since N is considered to be the residual content of TMAH of the raw material, the material of the free-standing membrane is considered to be composed of Ti, C, and O.

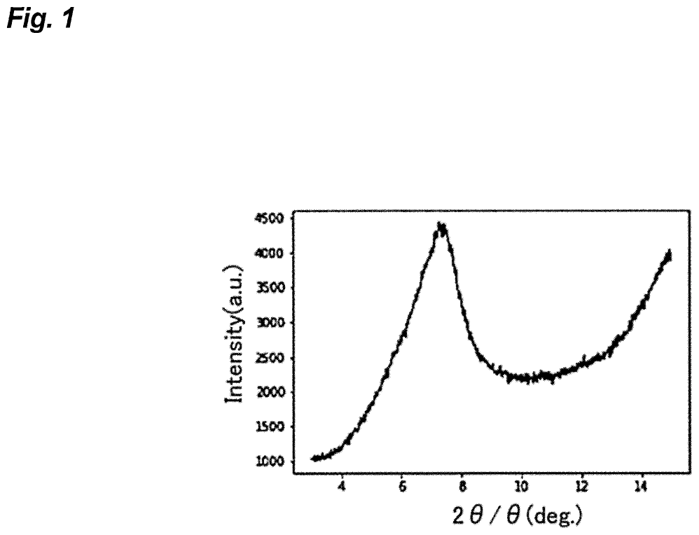

For the free-standing membrane obtained in the same manner as described above, an XRD profile was measured using an XRD apparatus (MiniFlex manufactured by Rigaku Corporation) (characteristic X-ray: CuKα=1.54 Å). The obtained XRD pattern is shown in FIG. 1. As understood from FIG. 1, this material had a peak at 2θ=7.26°.

### Preparation of Dry Powder

The sample slurry prepared above was dried by freeze drying (freezing in a freezer at −40° C., and then vacuum-drying). The resulting dry powder (corresponding to TiCO powder) was subjected to adsorption evaluation.

### Example 2

### Preparation of TiCO

First, a container (100 mL I Boy) was charged with 1.16 g of titanium boride (TiB₂, manufactured by Kojundo Chemical Laboratory Co., Ltd.) and 30 mL of a 25 mass % aqueous tetramethylammonium hydroxide (TMAH) solution (manufactured by Tokyo Chemical Industry Co., Ltd.). Thereto was placed a stirrer chip having a length substantially equal to the inner diameter of the circular bottom surface of the container (35 mm). While the container was kept at 50° C. in a water bath, the mixture in the container was stirred with the stirrer chip and maintained for 72 hours, thereby allowing the reaction to proceed. Next, the reaction mixture in the container was transferred to a 50 mL centrifuge tube with a stainless steel spatula (without the addition of a liquid medium such as ethanol or water). Centrifugation was performed using a centrifuge under conditions of 3500 G and 5 minutes to precipitate the solid content. (i) After centrifugation, the supernatant liquid was discarded, (ii) 40 mL of ethanol (manufactured by FUJIFILM Wako Pure Chemical Corporation) was added to the remaining precipitate in the centrifuge tube, and the mixture was subjected to handshake for 5 minutes (reslurry), and (iii) centrifugation was performed under the same conditions as described above. The operations (i) to (iii) were repeated until the pH of the supernatant liquid was 8 or less. When the operations were repeated three times, the pH of the supernatant liquid became 8 or less. Therefore, this supernatant liquid was discarded, and the repeated operations were terminated. 40 mL of pure water was added to the remaining precipitate in the centrifuge tube, and the mixture was shaken and stirred for 15 minutes using an automatic shaker. Thereafter, centrifugation was performed using a centrifuge under the conditions of 3500 G and 30 minutes, and the supernatant liquid was recovered as a sample slurry.

An analysis and preparation of dry powder were performed in the same manner as in Example 1.

### Example 3

### Preparation of TiCO

First, a vessel (100 mL I Boy) was charged with 10 g of titanium carbide (TiC, manufactured by Kojundo Chemical Laboratory Co., Ltd.) and 30 mL of a 25 mass % aqueous tetramethylammonium hydroxide (TMAH) solution (manufactured by Tokyo Chemical Industry Co., Ltd.). Thereto was placed a stirrer chip having a length substantially equal to the inner diameter of the circular bottom surface of the container (35 mm). While the container was kept at 50° C. in a water bath, the mixture in the container was stirred with the stirrer chip and maintained for 24 hours, thereby allowing the reaction to proceed. Next, the reaction mixture in the container was transferred to a 50 mL centrifuge tube with a stainless steel spatula (without the addition of a liquid medium such as ethanol or water). Centrifugation was performed using a centrifuge under conditions of 3500 G and 5 minutes to precipitate the solid content. After centrifugation, (i) the supernatant liquid was discarded, (ii) 40 mL of ethanol (manufactured by FUJIFILM Wako Pure Chemical Corporation) was added to the remaining precipitate in the centrifuge tube, and the mixture was subjected to handshake for 5 minutes (reslurry), and (iii) centrifugation was performed under the same conditions as described above. The operations (i) to (iii) were repeated until the pH of the supernatant liquid was 8 or less. When the operations were repeated three times, the pH of the supernatant liquid became 8 or less. Therefore, this supernatant liquid was discarded, and the repeated operations were terminated. 40 mL of pure water was added to the remaining precipitate in the centrifuge tube, and the mixture was shaken and stirred for 15 minutes using an automatic shaker. Thereafter, centrifugation was performed using a centrifuge under the conditions of 3500 G and 30 minutes, and the supernatant liquid was recovered as a sample slurry.

An Analysis and preparation of dry powder were performed in the same manner as in Example 1.

### Comparative Example 1

The spherical adsorption charcoal (“Kremezin disintegrating tablets 500 mg”, manufactured by Kureha Corporation) was powdered using a mortar and subjected to adsorption evaluation.

### Comparative Example 2

An adsorption type blood purifier (“Lixelle”, manufactured by KANEKA CORPORATION) was disassembled, and the extracted an adsorption body was subjected to adsorption evaluation.

### Comparative Example 3

A medicinal coal (manufactured by Nichi-Iko Pharmaceutical Co., Ltd.) was subjected to adsorption evaluation.

### Evaluation of Adsorption Performance

### Test 1: Adsorption of Na, P, K, β2-Microglobulin, Interleukin-18, α-Amylase, and Albumin

In a 50 mL centrifuge tube, 10 mL of human plasma collected from a healthy subject and 0.6 g of an adsorbent were weighed, and shaken and stirred for 15 minutes using a constant temperature shaker (TITEC BR-43FM) set at 37° C. Thereafter, the mixture was centrifuged in a centrifuge (TOMY AX-521) under conditions of 3200 rpm, 10 minutes, and 20° C., several mL of the supernatant was sampled, and component analysis was performed.

Component analysis was performed for Na, P, K, β2-microgloblin, interleukin-18 (IL-18), α-amylase, Albumin.

Na and K were measured by an electrode method; measurement of P and α-amylase was performed by an enzymatic method; measurement of β2-microglobulin was performed by a latex agglutination method; measurement of interleukin-18 was performed by an EIA method; and the albumin was measured by a colorimetric method (BGC method).

Note that the same treatment was also performed on the sample containing no adsorbent and containing only human plasma in the centrifuge tube, and the sample was used as a baseline of component concentration. The concentration of the component A contained in the human plasma after the adsorption test was performed was defined as CAI, and the concentration of the component A at the baseline was defined as CAO, and the adsorption removal rate was calculated based on the following formula:

$$Adsorption removal rate ( \% by mass ) = ( {C}_{A 0} - {C}_{A 1} ) / {C}_{A 0} \times 100$$

The results of this analysis are shown in Table 1.

TABLE 1
Adsorption removal rate for selected components.

| | Molecular | Example | Example | Example | Comparative | Comparative | Comparative |
|---|---|---|---|---|---|---|---|
| Component | weight | 1 | 2 | 3 | Example 1 | Example 2 | Example 3 |
| Na | 23 | 66 | 32 | 16 | 0 | 0 | 0 |
| P | 31 | 0 | 85 | 99 | 0 | 0 | 0 |
| K | 39 | 81 | 37 | 33 | 0 | 0 | 0 |
| β2-microglobulin | 11,800 | 80 | 20 | 0 | 0 | 99 | 5 |
| IL-18 | 18,000 | 0 | 0 | 2 | 0 | 0 | 0 |
| α-amylase | 54000 | 0 | 0 | 0 | 0 | 0 | 0 |
| Albumin | 66000 | 0 | 0 | 6 | 0 | 0 | 0 |

It was confirmed that the dry powder of Examples 1 to 3 exhibited an action of adsorbing a disease-causing substance, particularly Na and K (electrolyte) and β2-microglobulin (uremic substance). Thus, a pharmaceutical composition comprising MQO (TiCO in Example 1) may be useful for the treatment of a disease.

Test 2: Adsorption of Cytokines Other than Interleukin-18

As cytokine reagents, 10 ng of interferon-γ (INF-γ) (manufactured by FUJIFILM Wako Pure Chemical Corporation), 10 ng of interleukin-1β (IL-1β) (manufactured by FUJIFILM Wako Pure Chemical Corporation), 10 ng of interleukin-6 (IL-6) (manufactured by FUJIFILM Wako Pure Chemical Corporation), and 10 ng of a tumor necrosis factor (TNF-α) (manufactured by FUJIFILM Wako Pure Chemical Corporation), 0.5 mg of an adsorbent (dry powder of Example 2), and 20 mL of pure water were added in a 50 mL centrifuge tube, and shaken and stirred for 15 minutes using a constant temperature shaker (TITEC BR-43FM) set at 37° C. Thereafter, the mixture was centrifuged in a centrifuge (TOMY AX-521) under conditions of 4500 rpm, 5 minutes, and 20° C., several mL of the supernatant was sampled, and component analysis was performed.

In the component analysis, the absorbance of each of INF-γ, IL-1β, IL-6, and TNF-α was measured using an ELISA method (sandwich method), and the cytokine adsorption amount (concentration of cytokine+adsorbent) was determined using a calibration curve of the absorbance and the cytokine adsorption amount determined in advance.

Incidentally, the same treatment was also performed for those containing no adsorbent and containing only each cytokine reagent and water in the centrifuge tube, and used as a baseline of the component concentration. The concentration of the component A (concentration of cytokine+adsorbent) contained in the cytokine reagent-containing water after the adsorption test was performed was defined as C_{A1}, the concentration of the component A (concentration of only cytokine) at the baseline was defined as C_{A0}, and the adsorption removal rate was calculated based on the following formula:

$$Adsorption removal rate ( \% by mass ) = ( {C}_{A 0} - {C}_{A 1} ) / {C}_{A 0} \times 100$$

The results of this analysis are shown in Table 2.

TABLE 2
Adsorption removal rate (% by mass) for selected components.

Component

| | INF-γ | IL-1β | IL-6 | TNF-α |
|---|---|---|---|---|
| Adsorption removal | 69 | — | 21 | 6 |
| rate (% by mass) | | | | |

From Table 2, it was confirmed that MQO showed an action of adsorbing INF-γ, IL-6, and TNF-α. On the other hand, the effect of adsorbing IL-1β was not shown. From these, it can be said that MQO also has an effect of adsorbing INF-γ, IL-6, and TNF-α as cytokines, and a pharmaceutical composition containing MQO can be useful for treatment of a disease.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

1. A pharmaceutical composition comprising one or more nanofibers and/or nanoflakes of a material represented by the following formula:
MQₐO_{b}
wherein “M” is at least one element selected from the group consisting of Groups 3, 4, 5, 6, or 7,
wherein “Q” is at least one element selected from the group consisting of Groups 12, 13, 14, 15, or 16, provided that oxygen is excluded, and
wherein “a” is 0 or more and 2 or less,
wherein “b” is greater than 0 and 2 or less, and
wherein a crystal structure of the material is a lepidocrocite type.

2. The pharmaceutical composition according to claim 1, wherein the material has a peak at a diffraction angle 20 in a range of 2° or more and 12° or less in an X-ray diffraction pattern.

3. The pharmaceutical composition according to claim 1, wherein the “Q” is C, and the “a” is not 0.

4. The pharmaceutical composition according to claim 1, wherein the “M” is Ti, the “Q” is C, and the “a” is not 0.

5. A method for adsorbing a disease-causing substance in vivo, in a subject in need thereof, comprising:
administering the pharmaceutical composition according to claim 1 to the subject, thereby reducing the amount or concentration of the disease-causing substance within the subject, upon excretion of the composition according to claim 1 from the subject.

6. The method of claim 5, wherein the disease-causing substance comprises a uremic substance having a molecular weight of 100 or more.

7. The method of claim 6, wherein the uremic substance having a molecular weight of 100 or more comprises β2-microglobulin.

8. The method of claim 5, wherein the disease-causing substance comprises an electrolyte.

9. The method of claim 8, wherein the electrolyte comprises Na and/or K.

10. The method of claim 5, wherein the disease-causing substance comprises a cytokine.

11. The method of claim 10, wherein the cytokine comprises an interleukin, an interferon, a chemokine, a hematopoietic factor, a cell growth factor, and/or a tumor necrosis factor.

12. The pharmaceutical composition of claim 1, wherein the composition is formulated as an oral dosage form.

13. A method of treatment, comprising:
administering, to a subject in need thereof, an effective amount of nanofibers and/or nanoflakes of a material represented by the following formula:
MQₐO_{b}
wherein “M” is at least one element selected from the group consisting of Groups 3, 4, 5, 6, or 7,
wherein “Q” is at least one element selected from the group consisting of Groups 12, 13, 14, 15, or 16, provided that oxygen is excluded,
wherein “a” is 0 or more and 2 or less,
wherein “b” is greater than 0 and 2 or less, and
wherein a crystal structure of the material is a lepidocrocite type;
thereby reducing a concentration or amount of one or more disease-causing substances in the subject upon excretion of the nanofibers and/or nanoflakes from the subject.

14. The method of claim 13, wherein the material has a peak at a diffraction angle 2θ in a range of 2° or more and 12° or less in an X-ray diffraction pattern.

15. A method for treating or preventing one or more symptoms associated with a renal disease, the method comprising
administering, to a subject in need thereof, an effective amount of a composition comprising nanofibers and/or nanoflakes of a material represented by a following formula:
MQₐO_{b}
wherein “M” is at least one element selected from the group consisting of Groups 3, 4, 5, 6, or 7,
wherein “Q” is at least one element selected from the group consisting of Groups 12, 13, 14, 15, or 16, provided that oxygen is excluded,
wherein “a” is 0 or more and 2 or less,
wherein “b” is greater than 0 and 2 or less, and
wherein a crystal structure of the material is a lepidocrocite type;
thereby treating or preventing one or more symptoms associated with the renal disease.

16. The method of claim 15, wherein the material has a peak at a diffraction angle 2θ in a range of 2° or more and 12° or less in an X-ray diffraction pattern.

17. The method of claim 15, wherein the one or more symptoms associated with the renal disease comprise hypernatremia and/or hyperkalemia.
